(19) United States
(12) Patent Application Publication
(10) Pub. No.: US 2024/0133566 A1
(43) Pub. Date: Apr. 25, 2024

(54) Air Treatment Device

(71) Applicant: BELIMO Holding AG, Hinwil (CH)

(72) Inventors: Patrick REISSNER, Herrliberg (CH); Roman WYSS, St. Gallen (CH)

(73) Assignee: BELIMO Holding AG, Hinwil (CH)

(21) Appl. No.: 18/277,951

(22) PCT Filed: Feb. 24, 2022
(86) PCT No.: PCT/EP2022/054712

§ 371 Date: Aug. 17, 2023

(30) Foreign Application Priority Data
Feb. 25, 2021 (CH) 205/21

(51) Int. Cl. F24F 8/192; F24F 11/39; F24F 11/75; F24F 13/28

(52) U.S. Cl. CPC F24F 8/192; F24F 11/39; F24F 11/75; F24F 13/28; F24F 2110/50

(57) ABSTRACT

An air treatment device is disclosed. The air treatment device includes an air duct configured to deliver an air flow. A first filter stage is included and comprises at least one air filter arranged in the air duct for filtering the air flow. At least a second filter stage is also included and comprises at least one further air filter and is arranged downstream of the first filter stage (for filtering the air flow after having passed the first filter stage. The second filter stage is controllable between at least two states with different filtration efficiencies, whereby, with respect to a filtration efficiency in the first state, a filtration efficiency in the second state is lower, in particular the filtration efficiency in the second state is essentially zero. An air quality sensor is also present, the sensor allowing for measuring the air quality of the air flow in between the first and the second air filter stages.

### CROSS REFERENCE TO RELATED APPLICATIONS

This application is a national phase application of International Application No. PCT/EP2022/054712, filed Feb. 24, 2022 and which claims priority to Swiss patent application No. 205/21, filed Feb. 25, 2021, the content of both of which is herein incorporated by reference.

### BACKGROUND OF THE INVENTION

### Field of the Invention

The invention relates to an air treatment device as well as to an air handling unit (AHU) a rooftop unit (RTU), a fan coil and a heating, ventilating and air-conditioning (HVAC) system comprising the air treatment device. Furthermore, the invention is concerned with a method for filtering air.

### Description of Related Art

Air treatment devices are frequently used for cleaning outside air, e.g. before distributing it into buildings, or for reconditioning used for reintroducing it, usually together with admixed outside air, into a building. Air treatment devices can be stand-alone devices, which typically are combined with air handling units (AHU), rooftop units (RTU), fan coils, or heating, ventilating and air-conditioning (HVAC) systems of buildings. Alternatively, air treatment devices can be integrated in such units and systems.

Regardless of the specific application, air treatment devices typically comprise filter systems in order to provide air with a predefined purity or quality, respectively. Specifically, airborne pollutants or contaminants, respectively, such as e.g. dust particles and volatile organic compounds, are removed from the air with filtration devices. Although filtration devices are highly effective at removing undesired contaminants, they typically cause a significant pressure drop across the filters. The better the effectiveness or filtration efficiency of a filter device, the greater the pressure drop. Therefore, in order to compensate for the pressure loss, more fan energy must be utilized for keeping the air flow within the air treatment device at a desired level. This significantly increases energy consumption and operating costs.

For addressing these problems, there are systems that temporarily bypass one or more of the filter systems when the quality of the incoming air provided to the air treatment device is sufficiently good. This reduces the overall energy consumption of the air treatment device without sacrificing the quality of air discharged into air distribution systems of buildings.

In this regard, WO 96/23177 A1 (York International Corporation) describes an air conditioning unit with an initial filter and a high capacity filtration system for removing contaminants from supply air, typically return air which has been recirculated from the conditioned area. A filter bypass passage and suitable flow control devices are provided so that supply air, typically fresh outdoor air, may be routed around the high capacity filtration system when minimal or no filtration is required. Thereby, an air quality detector can be positioned upstream the initial filter or externally of the air conditioning unit, and the air quality detector can be coupled with a controller that controls the flow control device.

Likewise, FR 3 078 562 A1 (France Air SA) describes an air treatment device for the ventilation of buildings, the device comprising a fresh air inlet, for bringing in an incoming fresh air flow, and at least a filter, to filter the incoming fresh air flow. Furthermore, the device comprises a bypass of the filter, thanks to which at least part of the incoming fresh air flow can bypass the filter, as well as a closure member, to selectively close the bypass. Additionally, there is an electronic unit for controlling the closure member, able to collect information relating to the quality of the incoming fresh air and to control the closing member to open or close the bypass depending on the quality of the incoming fresh air.

However, as it turned out, also these systems have certain disadvantages. For example, heavily contaminated air can cause deposits on the air quality sensor affecting its functioning and the efficiency of the filter systems may change over time such that the bypass systems no longer function properly.

There is thus a need to provide improved solutions, which overcome the aforementioned drawbacks.

### BRIEF SUMMARY OF THE INVENTION

It is an object of the present invention to provide improved air treatment devices, especially capable of providing air with a predefined air quality or purity, respectively. In particular, the air treatment devices shall be capable of efficiently filtering incoming air with varying levels of contaminants with lowest possible energy consumption and costs. Furthermore, the air treatment devices should have a maintenance interval or service life, respectively, as long as possible.

Surprisingly, it has been found that the object of the invention can be solved with an air treatment device according to claim 1 as well as with a method for filtering air according to independent claim 14.

Thus, a first aspect of the present invention is directed to an air treatment device comprising:
a) an air duct for delivering an air flow;
b) a first filter stage comprising at least one air filter and being arranged in the air duct for filtering the air flow; at least a second filter stage comprising at least one further air filter and being arranged downstream of the first filter stage, for filtering the air flow after having passed the first filter stage;
c) whereby the second filter stage is controllable between at least two states with different filtration efficiencies, whereby, with respect to a filtration efficiency in the first state, a filtration efficiency in the second state is lower;
   and whereby, an air quality sensor is present, which allows for measuring the air quality of the air flow in between the first and the second filter stages.

While it is one possible embodiment, the inventive device is not restricted to a setup with only the air quality sensor in between the at least two filter stages. If desired, further components may be arranged in between the at least two filter stages as well, such as e.g. a fan, a heating unit, a cooling unit, a further sensor, a flow guiding element, a further filter and/or any other component.

However, in a preferred embodiment, viewed from the upstream end of the air duct, the at least one air filter of the first filter stage is the first filter in the air duct and the air quality sensor is arranged between the at least one filter of the first filter stage in the air duct and the at least one further air filter of the second filter stage without any further air filter and/or filter stage in-between.

“Filtration efficiency” is meant to be the ability of a filter stage to retain a given contaminant and/or to remove the contaminant form the air flow. A higher filtration efficiency indicates that the filter stage removes the contaminants more efficiently.

Especially, the filtration efficiency (E) can be expressed with the following formula: E=(1−c_{downstream}/cᵤₚₛₜᵣₑₐₘ); whereby c_{downstream} is the concentration of a given contaminant downstream the filter stage and cᵤₚₛₜᵣₑₐₘ is the concentration of the contaminant upstream the filter stage. Thus, the filtration efficiency can range from 0 (no filtration of contaminant at all) to 1 (complete filtration of contaminant).

The second filter stage is controllable between at least two states with different filtration efficiencies. Thereby, the term “at least two states” includes two states or more than two states, e.g. three states, four states or even more states.

The term controllable means switchable in a discrete manner between the at least two states with different filtration efficiencies as well as continuously adjustable between the at least to stages with different filtration efficiencies. Thus, the second filter stage is switchable or continuously adjustable.

If the second filter is switchable, it can only be present in one of the at least two states (which might be more than two states). Thereby, no intermediate state in between the at least two states can be selected.

If the second filter is continuously adjustable, it can be present in one of the at least two states as well as in any state in between the at least two states. In this case, for example, a state in between the at least two states can be set depending on the difference of the reading of the air quality sensor and the threshold.

The filtration efficiency is in particular related to an air contaminant. In particular, an air contaminant is a substance selected from carbon dioxide, carbon monoxide, sulfur oxides, nitrogen oxides, ammonia, ozone, particulates, toxic metals, radioactive substances, chlorofluorocarbons (CFCs), biological molecules, pollen, bacteria, viruses, volatile organic compounds, and/or hydrocarbon compounds, e.g. polycyclic aromatic hydrocarbon (PAH), especially particulates, in particular dust particulates. Thus, the second filter stage is in particular controllable between the at least two states with different filtration efficiencies related to one or more of these substances.

As it turned out, the inventive setup is highly beneficial in several respects: The first filter stage will remove at least a part of the contaminants, e.g. coarse dust, before the incoming air reaches the air quality sensor. Therefore, contamination of the air quality sensor is reduced, which in turn increases its lifetime compared to a situation in which the air quality sensor is placed upstream the first filter stage.

Also, the longer an air filter is used, the more particles, dust, fungi, etc. accumulate on its surface and in its volume. This typically increases the filtration efficiency of the at least one air filter in the first filter stage, so that with increasing time the second filter stage can be operated in the second state with lower filtration efficiency more often, resulting in additional energy savings. Likewise, if the efficiency of the at least one air filter in the first filter stage is reduced for whatever reasons, the second filter stage is kept in the first state with higher filtration efficiency more often so that the air provided by the air treatment device has a more constant quality. Put differently, the inventive setup inter alia allows for taking into account temporal variations of the filtration efficiency of the first filter stage as well as varying air qualities.

In contrast, when placing the air quality sensor upstream the first filter stage, such as in prior at solutions, this is not possible at all. In this case, for example, the system will unnecessarily close a bypass of the second filter stage although a first filter stage, due to an increased efficiency of its filter, already reduces the contaminants sufficiently. Overall, this results in a significantly higher energy consumption and shorter maintenance intervals.

Also, the air quality sensor can be used for monitoring and/or detecting abnormalities and/or to predict a time to a maintenance for the air filters.

Put differently, in a further preferred embodiment, the device is configured such that based on the information obtained from the air quality sensor, especially based on the temporal evolution of the reading of the air quality sensor, abnormalities are detected, a threshold for controlling the second filter stage is determined and/or a time to a maintenance for at least one of the air filters is predicted.

If, for example, the at least one air filter in the first filtration stage breaks down during operation, this can directly be detected with the air quality sensor. Specifically, in this case there will be a significant increase in the level of contaminants detected by the air quality sensor. Furthermore, if the first filter is clogged, the level of contaminants detected by the air quality sensor will likely decrease, e.g. be below an ordinary level, and/or the power required for a fluid flow machine to keep a given air velocity will increase.

Also, it is possible to use the air quality sensor to monitor the second air filter in the second filtration stage. When e.g. the filter class of the second filter is known, the time to the next maintenance can be estimated by extrapolation of the temporal evolution of contaminations measured by the air quality sensor.

Thus, for these cases, the air treatment device can be configured to display abnormalities and/or information about the state of the system. Furthermore, the air treatment device can be configured to send messages to an operator and/or to change the operating status of the air treatment device automatically.

Also, the information obtained with the air quality sensor, especially the temporal evolution of the reading of the air quality sensor, can be used for setting and/or adjusting the control of the second filter stage. For example, a threshold for switching between the at least two states can be set and/or adjusted based on the information obtained with the air quality sensor. Especially, if the reading of the air quality sensor is constantly increasing or decreasing over time, a threshold for controlling the second filter stage can for example be adjusted to operate the air treatment device more efficiently.

Consequently, information about the air quality downstream the first filter stage and upstream a controllable second filter stage is therefore highly beneficial and allows for setting up highly effective air treatment devices capable of providing air with a predefined air quality and having a reduced energy consumption and increased maintenance interval.

Especially, the air treatment device comprises a control unit which is configured such that if the reading of the air quality sensor is equal to or above a threshold, the second air filter stage is brought into a state with a filtration efficiency closer to the first state, switches to the first state or remains in the first state, whereas if the reading of the air quality sensor is below a threshold the second air filter stage is brought into a state with a filtration efficiency closer to the second state, switches to the second state or remains in the second state. Such a unit allows for automatically operating the device in optimal state.

In particular, the threshold can be a predefined threshold or an adjustable threshold. An adjustable threshold is meant to be a threshold which depends on a control parameter, e.g. the time in the day, the time elapsed since the last maintenance, the level of contamination of at least one of the air filter, the temporal evolution of the reading of the air quality sensor, a level of occupancy in the building, an external control parameter and/or an environmental parameter. An environmental parameter is for example selected from the temperature, the humidity and/or an air quality index outside the air treatment device, e.g. inside and/or outside a building. An external control parameter is for example provided by a centralized control unit.

Thus, the adjustable threshold may be set depending on one or more control parameter.

For example, the threshold might be set differently during daytime than during nighttime. Typically an office building is hardly occupied during nighttime. Consequently, the air quality can be kept at a lower level during nighttime which in turn helps to further reduce energy consumption.

A state with a filtration efficiency closer to the first state and/or a state with a filtration efficiency closer to the second state can be set depending on the difference of the reading of the air quality sensor and the threshold.

The control unit can be a stand-alone device or it can be integrated in one or more of the components of the air treatment device (see below).

However, in principle it is possible to run the air treatment device without such a control unit. In this case, the state of the second filter stage can for example be changed manually, depending on the reading of the air quality sensor.

According to a preferred embodiment, the filtration efficiency of the second filtration stage in the second state is at least 50%, in particular at least 90%, especially 100%, lower than the filtration efficiency in the first state. In particular, in the second state, the filtration efficiency of the second filtration stage is <0.5, especially <0.1, most preferred 0.

Especially, the air flow passes the second filter stage without being filtered, if the second filter stage is in the second state. In this case, filtration efficiency is zero resulting in a significantly reduced pressure drop across the second filter stage.

Thus, preferably, the second filter stage is configured such that (i) the air flow passes the at least one further filter of the second filter stage, if the second filter stage is in the first state and (ii) the air flow passes the second filter stage essentially without being filtered and/or without passing the at least one further filter, if the second filter stage is in the second state. In the latter case, the at least one further filter basically has no effect and the filtration efficiency is essentially zero resulting in a significantly reduced pressure drop across the second filter stage.

In particular, the at least one air filter of the first filter stage and the at least one further air filter of the second filter stage are located in a straight section of the air duct.

Especially preferred, the at least one further air filter of the second filter stage is mounted in the air duct such that the at least one further air filter is completely present inside the air guiding interior part of the air duct, independently of the state of the filter or in both states of the filter. Thereby, preferably, the at least one further air filter of the second filter stage is mounted in a straight section of the air duct.

Put differently, in this embodiment, the at least one further air filter preferably is completely integrated inside the air guiding interior part of the air duct, especially in a straight section of the air duct.

In particular, the at least one further air filter is completely present inside the air guiding interior part of the air duct in the first state, in which the air flow passes the at least one further filter of the second filter stage, as well as in the second state in which the air flow passes the second filter stage essentially without being filtered and/or without passing the at least one further filter.

These measures allow for implementing the inventive solution in a highly space saving manner directly in the air guiding interior part of the air duct.

Especially, in these cases, there is no bulge in the air duct for accommodating the at least one further air filter and/or there is no bypass for bypassing the at least one further air filter in the air duct.

In particular, the second air filter stage comprises a drive unit, especially a motor and/or an actuator, for moving and/or rotating the at least one further air filter of the second filter stage.

Especially, the second filter stage is configured for moving the at least one further air filter of the second air filter stage in a swinging movement.

Preferably, the drive unit allows for moving and/or rotating the at least one further air filter of the second filter stage from a first state, in which the air flow passes the at least one further filter of the second filter stage, to a second state in which the air flow passes the second filter stage essentially without being filtered and/or without passing the at least one further filter. The drive unit preferably is controlled by the above described control unit, if the latter is present.

According to a highly preferred embodiment, the second filter stage comprises a drive unit, especially a motor and/or an actuator, for moving and/or rotating the at least one further air filter of the second air filter stage from a first state, in which the at least one further air filter fills the duct cross-section, to a second state in which a free passage in the duct in parallel to the at least one further air filter is present in the duct. The drive unit preferably is controlled by the above described control unit, if the latter is present.

Put differently, in these embodiments, the at least one further air filter can simply be moved and/or rotated at least partly out of the air flow in the duct. This is a very reliable solution which can be implemented in a space saving manner directly in the duct.

In particular, the at least one further air filter of the second filter stage is rotatably mounted in the duct, such that the at least one further air filter is rotatable around an axis perpendicular to a longitudinal axis of the duct and/or a to a direction of air flow in the air duct.

Thereby, in particular, the at least one further air filter of the second filter stage is rotatably mounted on an axle and/or on a hinge in the duct.

Especially, the axle and/or a rotational axis of the hinge is arranged in a direction perpendicular to a longitudinal axis of the duct and/or to a direction of air flow in the duct.

Especially, the drive unit is configured to rotate the at least one further air filter of the second filter stage by >0°-90°, preferably 10°-90°, especially 45°-90°, in particular 85°-90°, most preferably 90°.

According to another highly preferred embodiment, the air treatment device comprises a controllable flow control device, which, if present in a first state, allows for routing the air flow having passed the first filter stage through the at least one further air filter of the second filter stage, and, if present in a second state, allows for routing the air flow having passed the first filter stage at least partially, preferably completely, around the at least one further filter of the second filter stage. The controllable flow control device preferably is controlled by the above described control unit, if the latter is present.

Especially, the flow control device comprises a flap and/or a valve.

In particular, the second air filter stage comprises a bypass, which is openable and closable with the flow control device, whereby in opened state, the bypass connects a region of the duct between the two filter stages and a region of the duct following the second air filter stage in downstream direction, especially such that a free passage in parallel to the second filter stage is present.

For example, the flow control device comprises a flap and/or a valve being arranged in parallel and/or adjacent to the at least one further air filter in the duct. Thereby, the at least one further air filter can be installed unmovable in a fixed manner in the duct. In this case, the cross-section of the duct in the region of the at least one further air filter is filled partly by the at least one further air filter and partly by the flow control device, if the latter is present in closed state. If the flow control device is in opened state, a free passage or bypass in parallel to the at least one further air filter is present in the duct. Therefore in this case, no separate conduct beside the duct is required for realizing the bypass.

However, in another preferred embodiment, the openable and closable bypass comprises a separate conduct running in parallel to the duct and which is openable and closable with the flow control device, especially at an inlet side. In this case, the at least one further air filter can completely fill the cross-section of the duct. Also, the at least one further air filter can be installed unmovable in a in a fixed manner in the duct.

In another beneficial embodiment, the at least one further air filter of the second filter stage is placed in the separate conduct and the flow control device, especially a flap and/or valve, is arranged such that either the duct or the separate conduct can be closed or opened. This allows for providing the second filter stage in the form of an add-on element, which can easily be attached to an existing duct.

In particular, for all of the above mentioned embodiments, a size of the cross-section of the free passage in parallel to the second filter stage preferably is at least 5%, in particular at least 30%, especially at least 50%, of the size of the cross-section of the duct in the region of the second filter stage.

According to a further preferred embodiment, the at least one further filter of the second filter stage is a filter with controllable filtration efficiency. Thereby preferably, the air treatment device is configured such that the filtration efficiency of the filter is controlled depending on the reading of the air quality sensor. The filter with controllable filtration efficiency preferably is controlled by the above described control unit, if the latter is present.

A filter with controllable filtration efficiency for example is a filter that changes the properties of the filter medium by an external stimulus, e.g. a mechanical action, an electrical field, an electrical current and/or a magnetic field in the air flow. Especially, the air quality sensor is a sensor for determining a proportion of particulates, in particular dust particulates, in the air flow. However, depending on the application, sensors for detecting other contaminants are suitable as well.

In particular, the air quality sensor is a particulate sensor. Especially the particulate sensor is configured for detecting particulates with a particle size of 0.1-20 μm, in particular 0.1-10 μm, especially 0.1-1 μm. Such sensors allow for detecting dust contaminants which typically are present in environmental air.

Especially the particulate sensor comprises a light source emitting electromagnetic waves with wavelength from 100 nm-10,000 nm, a detection area and an optoelectronic component, e.g. a photodiode, for detecting light scattered on particles passing the detection area. Such sensors turned out to be highly reliable for the inventive purposes.

Nevertheless, other sensors might be suitable as well.

Especially, the air quality sensor is arranged in between the first filter stage and the second filter stage. However, if there are more filter stages, one or more air quality sensors can be arranged between successive filters stages.

Preferably, the at least one air filter and/or the at least one further air filter are selected from particulate filters, molecular filters, catalytic filters, electrostatic filters, vortex filters, surface deposition filters, and/or electromagnetic wave filters, e.g. ultraviolet radiation filters.

Especially at least one air filter and/or the at least one further air filter are chosen from filters having a filter medium which is flowed through by the air flow. Most preferably, the filters are chosen from particulate filters and/or molecular filters.

In other preferred embodiments, the at least one air filter and/or the at least one further air filter is an electrostatic filter. Such filters are also known as electrostatic precipitators (ESP). These are filtration device that remove particulates, e.g. like dust, from an air flow using the force of an induced electrostatic charge.

Preferably, the particulate filter is a coarse dust filter for particulates >10 μm, a fine dust filter for particulates <10 μm, a fine dust filter for particulates <2.5 μm, a fine dust filter for particulates <1 μm, an efficient particular air filter (EPA filter), a high-efficiency particulate air filter (HEPA filter) and/or an ultra low penetrating air filter (ULPA) for particulates with sizes of <0.5 μm, in particular with sizes of 0.001-0.3 μm.

The particulate filter in particular is a filter of class ISO coarse, ISO ePM10, ISO ePM2.5, ISO ePM1, as classified in EN ISO 16890-1:2017, and/or a filter of class E10-U17, according to DIN EN 1822-1:2019.

Such particulate filters are highly beneficial for filtering contaminants which typically are present in environmental air.

In particular, the molecular filter is a carbon filter comprising activated carbon, e.g. carbon with a surface area of >3,000 m²/g. Carbon filters are highly versatile and allow for filtering many different molecular compounds typically present in environmental air. However, other molecular filters might be suitable as well.

Especially, the at least one air filter of the first filter stage is a particulate filter and the at least one further air filter of the second filter stage is a molecular filter. Such a setup is highly beneficial if beside dust particles, varying amounts of chemical contaminants are present in the incoming air.

According to another preferred embodiment, the at least one air filter of the first filter stage and the at least one further air filter of the second filter stage both are particulate filters, whereby, preferably the second filter stage is configured for filtering smaller particles than the first filter stage. This setup is for example recommended if varying amounts of dust particles with different sizes are present in the incoming air.

However, other combinations of filters might be suitable as well for other applications.

Preferably, the air treatment device additionally comprises a fluid-flow machine, especially a fan, for generating an air flow in the duct. The fluid-flow machine preferably is controlled by the above described control unit, if the latter is present.

Furthermore, the air treatment device comprises an air velocity probe. Such a probe allows for measuring the velocity of the air flow in the duct. The air velocity probe preferably is connected to the above described control unit, if the latter is present.

Preferably, the control unit, if present, is configured such that the fluid-flow machine is controlled so that an air velocity of the air flow in the duct is essentially constant, especially independently of the state of the second filter stage. This allows for keeping the air velocity in the duct in a range that is optimal for filtering, e.g. 0.1-10 m/s, especially 1-3 m/s. This can for example be realized with a feedback controller using the power of the fluid-flow machine as a control.

Nevertheless, such a control unit is not mandatory. It is in principle possible as well, to run the fluid-flow machine with constant power if varying air velocities or flow rates are not an issue.

Especially, the air treatment device comprises a control unit as described above. In particular, control unit is integrated in the air quality sensor, the drive unit and/or the flow control device. However, the control unit can be provided as standalone unit as well.

It should be noted that the air treatment device is not restricted to embodiments with only two filter stages. The device may comprise further filter stages, especially downstream the second filter stage.

Preferably, at least one or all of the further filter stages are controllable between at least two states with different filtration efficiencies, whereby, with respect to a filtration efficiency in the first state, a filtration efficiency in the second state is lower. Put differently, the at least one or all of the further filter stages preferably function like the second filter stage.

Thereby, preferably, at least one further air quality sensor is present, which allows for measuring the air quality of the air flow in between the second and the at least one further filter stage and/or between any other further filter stages.

Preferably, at least one or all of the further filter stages are configured as described above for the second filter stage and/or are controlled by the control unit as described above for the second filter stage.

Such a setup with more than two filter stages has the advantage that filtering of contaminants can be controlled even more precisely and the energy consumption can further be optimized.

A further aspect of the present invention is directed to an air handing unit, a rooftop unit, a fan coil, a heating system, a ventilating system, and/or an air-conditioning system, especially for a building, comprising an air treatment device as described above. Especially, the air treatment device is configured as an air handing unit, in particular as a roof top unit. The latter can be installed on a roof of a building.

Especially, the unit or system comprises an inlet for supplying fresh air, especially form the outside of a building, to an upstream side of the first filter stage of the air treatment device and an outlet for discharging air having passed the air treatment device, especially into a building.

Optionally there is a return line for discharging used air, e.g. form the inside of a building, through an exhaust, and a bypass for discharging the used air back to the inlet and/or the upstream side of the air treatment device, whereby, preferably, there is a regulating unit for controlling the mixing of fresh air and used air before reintroduction into the air treatment device.

Especially preferred, the unit or system is arranged in a building such that the inlet for supplying fresh air opens to the outside of the building and the outlet for discharging air having passed the air treatment device, opens into an interior part of the building, especially into a room of the building.

With such an arrangement, outside air from the outside of a building can be drawn in for filtering the outside air and supplying it into the building.

Thus, a further aspect of the present invention is directed to a building having a respective unit or system as described above.

Another aspect of the present invention is directed to a method for filtering air, especially with an air treatment device as described above, comprising the steps of:
a) delivering an air flow with an air duct having a first filter stage being arranged in the duct and comprising at least one air filter, and a second filter stage, downstream of the first air filter, comprising at least one further air filter; whereby the second air filter stage is controllable between at least two states with different filtration efficiencies, whereby, with respect to a filtration efficiency in the first state, a filtration efficiency in the second state is lower;
b) filtering the air flow with the first filter stage;
c) measuring the air quality in the air flow between the two filter stages with an air quality sensor;
d) comparing the reading of the quality sensor with a given threshold and:
   if the reading of the air quality senor is equal to or above a threshold, bringing the second filter stage into a state with a filtration efficiency closer to the first state, switching the second filter stage to the first state or keeping it in the first state; or
   if the reading of the air quality sensor is below the threshold, bringing the second filter stage in a state with a filtration efficiency closer to the second state, switching the second filter stage to the second state or keeping it in the second state.

Advantages described above in connection with the inventive air treatment device and its specific embodiments are likewise given with regard to the inventive method.

Preferably, the method is performed with the inventive air treatment device, which in particular has a control unit as described above.

Advantageously, the air flow in the duct is generated with a fluid-flow machine, especially a fan, and an air velocity of the air flow in the duct is determined with an air velocity probe.

Thereby, preferably, the fluid-flow machine is controlled such that the air velocity of the air flow in the duct is essentially constant, especially independently of the state of the second filter stage. For example, the air velocity of the air flow in the duct is kept constant with a feedback controller using the power of the fluid-flow machine as a control.

In particular, based on the reading of the air quality sensor, especially based on the temporal evolution of the reading of the air quality sensor, the control of the second filter stage is set and/or adjusted. For example, a threshold for switching between the at least two states is set based on the information obtained with the air quality sensor. Especially, if the reading of the air quality sensor is constantly increasing or decreasing over time, a threshold for controlling the second filter stage is for example adjusted to operate the air treatment device more efficiently.

In a further preferred implementation, based on the reading of the air quality sensor, especially based on the temporal evolution of the reading of the air quality sensor, abnormalities are detected, a threshold for controlling the second filter stage is determined and/or a time to a maintenance for one of the air filters is predicted.

An air flow in the duct preferably is from 0.1-10 m/s, especially 1-3 m/s.

Especially preferred, the method is a method of filtering outside air drawn in from the outside of a building. Put differently, the air flow delivered in step a) preferably comprises or consists of outside air from the outside of a building.

Thereby, preferably, the outside air or the air flow delivered in step a) at first is filtered with the first filter stage before entering the building.

Further advantageous configurations of the invention are evident from the exemplary embodiments.

### BRIEF DESCRIPTION OF THE SEVERAL VIEWS OF THE DRAWINGS

Further advantages features and details of the various embodiments of this disclosure will become apparent from the ensuing description of a preferred exemplary embodiment or embodiments and further with the aid of the drawings. The features and combinations of features recited below in the description, as well as the features and feature combination shown after that in the drawing description or in the drawings alone, may be used not only in the particular combination recited but also in other combinations on their own without departing from the scope of the disclosure.

The following is an advantageous embodiment of the invention with reference to the accompanying figures, wherein:

FIG. 1 depict a cross-section along the longitudinal direction of a first air treatment device with an air quality sensor between a first and a second filter stage and a flap-based bypass system next to the air filter of the second filter stage with the flap in closed state;

FIG. 2 depicts the device of FIG. 1 with the flap-based bypass system in opened state;

FIG. 3 depicts a cross-section along the longitudinal direction of a second air treatment device having a rotatably mounted further air filter in closed state;

FIG. 4 depicts the unit of FIG. 3 with the further air filter rotated into the open state;

FIG. 5 depicts a cross-section along the longitudinal direction of a third air treatment device, which has two filter stages arranged in series downstream the air quality sensor;

FIG. 6 depicts a cross-section along the longitudinal direction of a fourth air treatment device having a second filter stage with a separate bypass line around the further filter, whereby the bypass line is closable and openable with a flap;

FIG. 7 depicts a cross-section along the longitudinal direction of a fifth air treatment device having a second filter stage with a separate bypass line comprising the further air filter, whereby a flap allows for closing either the bypass line or the duct; and

FIG. 8 depicts an air handling unit comprising the air treatment device of FIG. 1.

In the figures, the same components are given the same reference symbols.

### DETAILED DESCRIPTION OF THE INVENTION

As used throughout the present disclosure, unless specifically stated otherwise, the term “or” encompasses all possible combinations, except where infeasible. For example, the expression “A or B” shall mean A alone, B alone, or A and B together. If it is stated that a component includes “A, B, or C”, then, unless specifically stated otherwise or infeasible, the component may include A, or B, or C, or A and B, or A and C, or B and C, or A and B and C. Expressions such as “at least one of” do not necessarily modify an entirety of the following list and do not necessarily modify each member of the list, such that “at least one of “A, B, and C” should be understood as including only one of A, only one of B, only one of C, or any combination of A, B, and C.

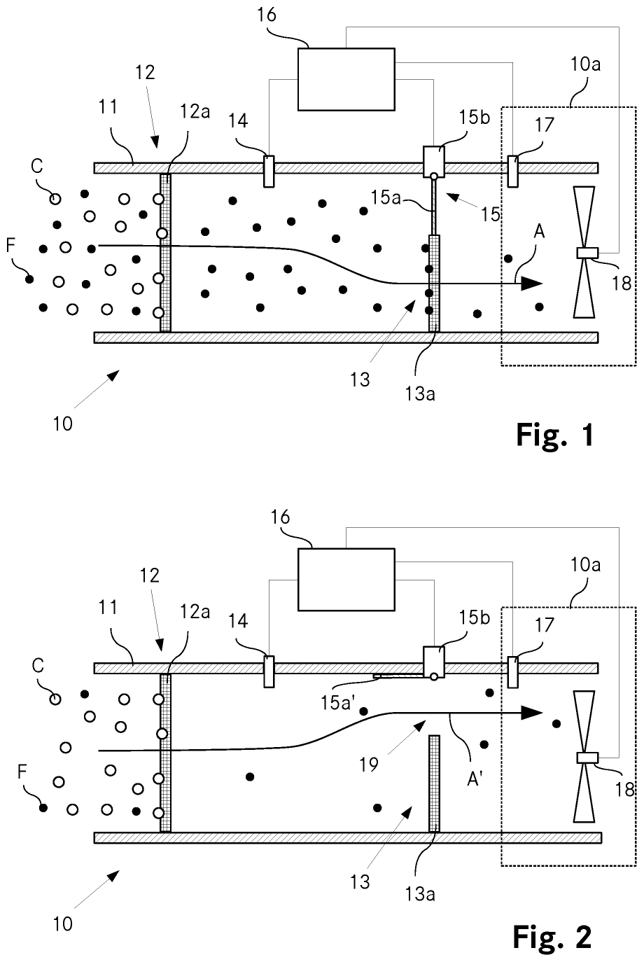

FIG. 1 shows a cross-section along the longitudinal direction of a first air treatment device 10. The device 10 comprises a duct 11 with rectangular cross-section with a first filter stage 12 consisting of an air filter 12a arranged in an upstream section and a second filter stage 13 comprising a further air filter 13a arranged in a downstream section of the duct 11. For example, the first air filter 12a is a particulate filter for coarse particulates (>10 μm), whereas the further air filter 13a is a particulate filter for fine particulates (<1 μm).

The second air filter 13a covers only a part of the cross-section of the duct 11. The remaining part of the cross-section is covered by a flap 15a, which is part of a controllable flow control device 15. The flap 15a can be rotated around a hinge with a drive unit 15b, for example a motor or an actuator.

Furthermore, the air treatment device 10 comprises an air quality sensor 14 being arranged in the duct 11 between the two filter stages 12, 13. The air quality sensor 14 is for example a particulate sensor comprising a light source emitting electromagnetic waves with wavelength from 100 nm-10,000 nm and a photodiode for detecting light scattered on particles passing the detection area of the particulate sensor within the duct 11.

The air quality sensor 14 is connected to a control unit 16 which is configured such that if the reading of the air quality sensor 14 is equal to or above a predefined threshold, the flow control device 15 switches to a first state or remains in the first state in which the flap 15a is in the closed position as shown in FIG. 1, whereas if the reading of the air quality sensor 14 is below a predefined threshold, the flow control device 15 switches to the second state or remains in the second state in which the flap 15a is brought into an open position with the drive unit 15b (cf. FIG. 2).

Additionally, there are a fluid-flow machine 18 for generating an air flow A within the duct and an air velocity probe 17 connected to the control unit 16 and capable of measuring the air velocity within the duct 11 (surrounded by a dashed rectangle). The control unit 16 is configured such that the air velocity of the air flow A in the duct can be kept constant, e.g. around 1.5 m/s, with a feedback controller using the power of the fluid-flow machine 18 as a control.

The fluid-flow machine 18 for generating an air flow A within the duct and the air velocity probe 17 typically are part of a ventilation unit 10a which is separate from the other components of the first air treatment device 10. Thus, for example, the air treatment device 10 can be a retrofit unit which can be combined with a ventilation unit 10a already present in the duct. However, it is also possible that the fluid-flow machine 18 for generating an air flow A within the duct and the air velocity probe 17 both are an integral component of the air treatment device 10.

FIG. 1 shows a situation in which incoming air comprises contaminants in the form of coarse dust particles C as well as fine dust particles F. Upon passing the first filter 12a, coarse particles C are filtered off whereas most of the fine particles F reach the downstream side of the first filter stage 12. Since the concentration of particles detected by the air quality sensor 14 is above a predefined threshold, the control unit 16 keeps the controllable flow control device 15 in closed state, i.e. the flap 15a is closed (=first state). This causes the air flow A to pass through the further air filter 13a which filters off most of the fine particles F from the air flow A. Thus, the air flow A at the downstream side of the second filter stage 13 comprises an acceptable concentration of contaminants.

FIG. 2 shows the situation in which the incoming air has a different level of contaminants when compared to the situation shown in FIG. 1. Although the incoming air comprises a comparable concentration of coarse particles C, the concentration of fine particles F in the situation shown in FIG. 2 is rather low. Since in this case the concentration of particles detected by the air quality sensor 14 is below the predefined threshold, the control unit 16 keeps the controllable flow control device 15 in opened state, i.e. the flap is open (denoted as 15a′; =second state), such that next to the second air filter 13a there is a free passage 19 in the second filter stage 13. This causes the air flow N to pass around the further air filter 13a through the free passage 19 since the flow resistance is much lower along this path. In this state the filtration efficiency of the second filtration stage 13 is essentially zero, i.e. lower than in the situation shown in FIG. 1.

Similar to the situation shown in FIG. 1, the air flow N at the downstream side of the second filter stage 13 comprises an acceptable concentration of contaminants although the further air filter 13a is bypassed. However, thanks to the reduced pressure loss, the power of the fluid-flow machine 18 can be reduced while maintaining the same air velocity.

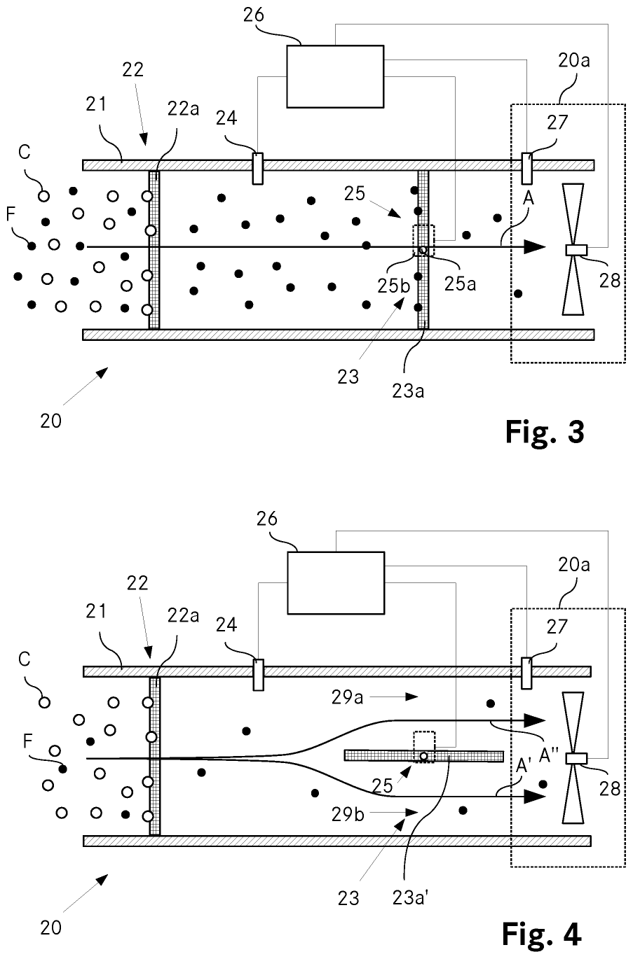

FIG. 3 shows a second air treatment device 20. The second device 20 comprises a duct 21, a first filter stage 22 with an air filter 22a, an air quality sensor 24, a control unit 26, an air velocity probe 27 and a fluid-flow machine 28, which are essentially identical in construction with the corresponding elements shown in FIG. 1. Also in this embodiment, the fluid-flow machine 28 for generating an air flow A within the duct and the air velocity probe 27 typically are part of a ventilation unit 20a which is separate from the other components of the second air treatment device 20.

However, in the embodiment shown in FIG. 3, the second filter stage 23 comprises a further air filter 23a which is rotatably mounted on an axle 25a at half height of the duct 21. The second air filter 23a can be rotated around the axle 25a with a drive unit 25b. Thus, in this case, the flow control device 15 comprises the second air filter 23a together with axle 25 and drive unit 25b. In the situation shown in FIG. 3, the flow control device is in a first state in which the cross-section of duct 21 is completely filled with the second air filter 23a, such that the air flow A, after having passed the first air filter 22 is additionally passing through the second air filter 23a of the second filter stage 23.

FIG. 3 shows a situation in which incoming air comprises a relatively high concentration of contaminants in the form of coarse particles C as well as fine particles F, similar to the situation shown in FIG. 1. However, due to the filtering at the first filter stage 22 and the second filter 23a, the air flow A at the downstream side of the second filter stage 23 comprises an acceptable concentration of contaminants.

FIG. 4 shows the second air handling unit 20 with the flow control device 25 in a second state, whereby the second air filter is rotated by about 90° (=opened second air filter 23a′) when compared to the situation shown in FIG. 3. In this state, there is an upper free passage 29a above the opened second air filter 23a′ and a lower free passage 29b below the opened second air filter 23a′.

With regard to the incoming air, the situation shown in FIG. 4 is comparable to the situation shown in FIG. 2, i.e. although the incoming air comprises a comparable concentration of coarse particles C, the concentration of fine particles F in the situation shown in FIG. 4 is rather low. Since In this case the concentration of particles detected by the air quality sensor 24 is below the predefined threshold, the control unit 26 keeps the controllable flow control device 25 in opened state, i.e. the second air filter 23a′ is open. This causes the air flow to split into two air flows A′, A″, passing around the second air filter 23a′ through the two free passages 29a, 29b.

Similar to the situation shown in FIG. 3, the air flows A′, A″ at the downstream side of the second filter stage 23 comprise an acceptable concentration of contaminants although the second air filter 23a′ is bypassed. Thanks to the reduced pressure loss, the power of the fluid-flow machine 28 can be reduced while maintaining the same air velocity.

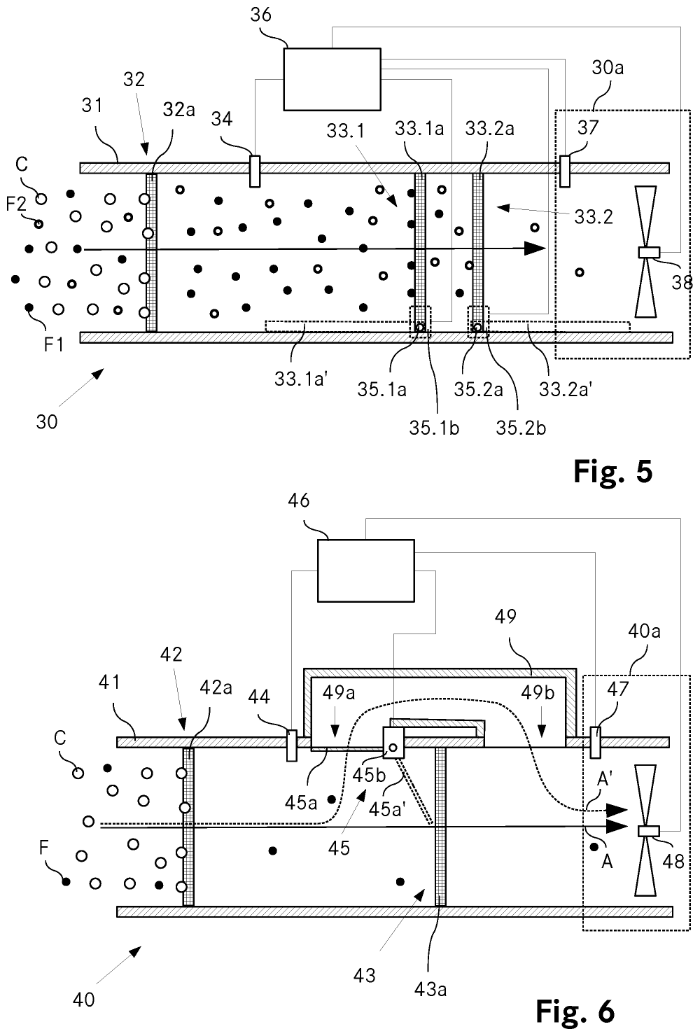

FIG. 5 shows a third air treatment device 30. The third device 30 comprises a duct 31, a first filter stage 32 with an air filter 32a, an air quality sensor 34, a control unit 36, an air velocity probe 37 and a fluid-flow machine 38, which are essentially identical in construction with the corresponding elements shown in FIG. 1. Also in this embodiment, the fluid-flow machine 38 for generating an air flow A within the duct and the air velocity probe 37 typically are part of a ventilation unit 30a which is separate from the other components of the second air treatment device 30.

However, in the embodiment shown in FIG. 5, the second filter stage 33.1 comprises an air filter 31.1a which is rotatably mounted on a hinge 35.1a at the bottom of the duct 31, so that second air filter 33.1a can be rotated counterclockwise with a first drive unit 35.1a into an open position in which the second air filter lies on the bottom of the duct 31 (=opened air filter 33.1a′, indicated by dashed outline).

Additionally, there is a third filter stage 33.2 comprising another air filter 33.2a which is rotatably mounted on another hinge 35.2a at the bottom of the duct 31, so that the air filter 33.2a can be rotated clockwise with a second drive unit 35.2a into an open position in which the air filter lies on the bottom of the duct 31 (=opened air filter 33.2a′, indicated by dashed outline).

Both, the second and the third filter stages 33.1, 33.2 are controlled with the control unit 36. In case of heavily contaminated incoming air, e.g. comprising different particulates, e.g. coarse particles C, fine particles F1 with a large diameter and fine particles F2 with a small diameter, the air flow, after having passed the first filter stage 32 passed both filters 33.1a, 32.2a. Thereby, the filter 33.1a filters out fine particles F1, whereas the filter 33.2a filters out fine particles F2.

If, however, the level of fine particles F2 as detected by the air quality sensor 34 is below a given threshold, the control unit 36 opens filter 33.2a in order to reduce the flow resistance. Likewise if the level of fine particles F1 as detected by the air quality sensor 34 is below a given threshold, the control unit 36 opens the filter 33.1a. Thereby, the filters 33.1a, 33.2a can be opened and closed independently such that, depending on the quality the air having passed the first filter stage 32, the flow resistance and thus the energy consumption of the fluid-flow machine 38 at a given air velocity in the duct 31 is minimized.

FIG. 6 shows a fourth air treatment device 40. The fourth device 40 comprises a duct 41, a first filter stage 42 with an air filter 42a, an air quality sensor 44, a control unit 46, an air velocity probe 47 and a fluid-flow machine 48, which are essentially identical in construction with the corresponding elements shown in FIG. 1. Also in this embodiment, the fluid-flow machine 48 for generating an air flow A within the duct and the air velocity probe 47 typically are part of a ventilation unit 40a which is separate from the other components of the second air treatment device 40.

However, in the embodiment shown in FIG. 6, the second filter stage 43 comprises a fixed filter 43a completely filling the cross-section of the duct 41. Unlike the other devices 10, 20, 30, the fourth device in addition comprises a separate bypass line 49 running in parallel to the duct 41, with a lateral opening 49a at the upstream side of the second filter stage 43 and a lateral opening 49b at the downstream side of the second filter stage 43.

The opening 49a at the upstream side can be closed and opened with a flap 45a (flap in opened state is indicated by dashed outline and denoted as 45′), which is driven by a drive unit 45b. Flap 45a and drive unit 45b form a flow control device 45, which is driven by the control unit 46 and allows for routing the air flow having passed the first filter stage 42 either through the further filter 43a (=air flow A) or around the further filter 43a through the bypass line 49 (=air flow A′, indicated by dashed line). Otherwise the functioning of the fourth air handling unit 40 is essentially identical with the first air handling unit 10.

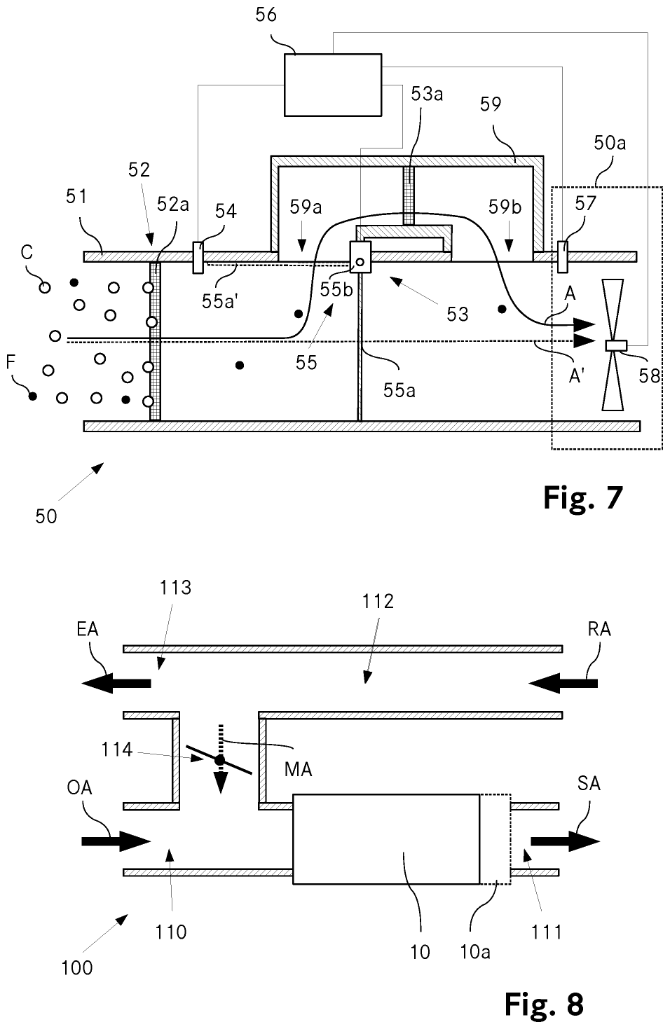

FIG. 7 shows a fifth air treatment device 50. The fifth device 50 comprises a duct 51, a first filter stage 52 with an air filter 52a, an air quality sensor 54, a control unit 56, an air velocity probe 57, a bypass-line 59 and a fluid-flow machine 59, which are essentially identical in construction with the corresponding elements shown in FIG. 6. Also in this embodiment, the fluid-flow machine 58 for generating an air flow A within the duct and the air velocity probe 57 typically are part of a ventilation unit 50a which is separate from the other components of the second air treatment device 50.

However, in the embodiment shown in FIG. 7, the further filter 53a of the second filter stage 53 is arranged in the bypass-line 59 with a lateral opening 59a at the upstream side of the second filter stage 53 and a lateral opening 59b at the downstream side of the second filter stage 53.

The opening 59a at the upstream side can be closed and opened with a flap 55a (flap in opened state is indicated by dashed outline and denoted as 55a′), which is driven by a drive unit 55b. Flap 55a and drive unit 55b form a flow control device 55, which is driven by the control unit 56. The flap 55a can either close the duct 51 along its longitudinal axis (in this case the opening 59a of the bypass-line 59 is open) or close the opening 59a of the bypass-line 59 (denotes as 55a′; in this case duct 51 is closed along the longitudinal axis at the second filter stage 53). Thus, the flow control device 55 allows for routing the air flow having passed the first filter stage 52 either through the bypass-line 59 and the further filter 53a (=air flow A) or around the further filter 53a directly along the longitudinal axis of duct 51 (=air flow A′, indicated by dashed line). Otherwise the functioning of the fourth air handling unit 50 is essentially identical with the first air handling unit 10.

FIG. 8 show an air handling unit 100 comprising the air treatment device 10 of FIG. 1 (or any of the other air treatment devices 20, 30, 40, 50). The unit 100 comprises an inlet line 110 for supplying outside air OA to the upstream side of the air treatment device 10. At the downstream side of device 10, an outlet 111 for discharging air having passed the air treatment device into a ventilation system of a building (not shown) is present.

Furthermore, there is a return line 112 for discharging return air RA from the building either via an exhaust 113 as exhaust air EA and/or for mixing return air RA via a bypass 114 with a regulating unit, e.g. a valve, with outside OA before reintroduction into the air treatment device 10.

Thus, it will be appreciated by those skilled in the art that the present invention can be embodied in other specific forms without departing from the spirit or essential characteristics thereof. The presently disclosed embodiments are therefore considered in all respects to be illustrative and not restricted.

In summary, it is to be noted that the invention provides highly beneficial air treatment devices that can be operated with minimized energy consumption while constantly providing an air stream with a desired level of contaminants. Furthermore, the air treatment devices show an improved service and maintenance life.

1. Air treatment device comprising:
a) an air duct for delivering an air flow;
b) a first filter stage comprising at least one air filter and arranged in the air duct for filtering the air flow;
c) at least a second filter stage comprising at least one further air filter and arranged downstream of the first filter stage, for filtering the air flow after having passed the first filter stage;
d) wherein the second filter stage is controllable between at least two states with different filtration efficiencies, such that, with respect to a filtration efficiency in the first state, a filtration efficiency in the second state is lower the filtration efficiency in the second state is essentially zero; and
e) the air treatment device further comprises an air quality sensor configured to measure the air quality of the air flow in between the first and the second air filter stages.

2. The air treatment device according to claim 1, whereby the device comprises a control unit which is configured such that if the reading of the air quality sensor is equal to or above a threshold, the second air filter stage is brought into a state with a filtration efficiency closer to the first state, switches to the first state or remains in the first state; whereas if the reading of the air quality sensor is below a threshold the second air filter stage is brought into a state with a filtration efficiency closer to the second state, switches to the second state or remains in the second state.

3. The air treatment device according to claim 1, wherein the second filter stage is configured such that:
(i) the air flow passes the at least one further filter of the second filter stage, if the second filter stage is in the first state; and
(ii) the air flow passes the second filter stage essentially without being filtered and/or without passing the at least one further filter, if the second filter stage is in the second state.

4. The air treatment device according to claim 1, wherein the second air filter stage comprises a drive unit for moving and/or rotating the at least one further air filter of the second filter stage from a first state, in which the air flow passes the at least one further filter of the second filter stage, to a second state in which the air flow passes the second filter stage essentially without being filtered and/or without passing the at least one further filter.

5. The air treatment device according to claim 4, wherein the at least one further air filter of the second filter stage is rotatably mounted in the duct, such that the at least one further air filter is rotatable around an axis perpendicular to a longitudinal axis of the duct and/or perpendicular to a direction of air flow in the air duct.

6. The air treatment device according to claim 1, further comprising a controllable flow control device configured so as to, if present in a first state, allows for routing the air flow having passed the first filter stage through the at least one further air filter of the second filter stage and, if present in a second state, allows for routing the flow having passed the first filter stage partially or completely, around the at least one further filter of the second filter stage.

7. The air treatment device according to claim 6, whereby the second air filter stage comprises a bypass, which is openable and closable with the flow control device, whereby in opened state, the bypass connects a region of the duct between the two filter stages and a region of the duct following the second air filter stage in downstream direction.

8. The air treatment device according to claim 6, wherein the flow control device comprises at least one of a flap and a valve being arranged in at least one of parallel and adjacent to the at least one further air filter in the duct.

9. The air treatment device according to claim 1, wherein,
the at least one further filter of the second filter stage is a tilter with adjustable filtration efficiency, e.g. an electrostatic filter; and
the air quality sensor is a sensor for determining a proportion of carbon dioxide, carbon monoxide, sulfur oxides, nitrogen oxides, ammonia, ozone, particulates, toxic metals radioactive substances, chlorofluorocarbons (CFCs), biological molecules, pollen, bacteria, viruses, volatile organic compounds, and/or hydrocarbon compounds in the air flow.

10. (canceled)

11. The air treatment device according to claim 1, wherein at least one of the at least one air filter and the at least one further air filter comprise at least one of particulate filters, molecular filters, catalytic filters, photocatalytic purifiers, plasma purifiers, water washing purifiers, electrostatic filters, vortex filters, surface deposition filters, and electromagnetic wave filters.

12. The air treatment device according to claim 11, wherein the at least one air filter of the first filter stage and the at least one further air filter of the second filter stage both are particulate filters, wherein the second filter stage is configured for filtering smaller particles than the first filter stage.

13. (canceled)

14. The air treatment device according to claim 1, wherein, when viewed from the upstream end of the air duct, the at least one air filter of the first filter stage is the first filter in the air duct and the air quality sensor is arranged between the at least one filter of the first filter stage in the air duct and the at least one further air filter of the second filter stage without at least one of any further air filter and any further filter stage in-between.

15. The air treatment device according to claim 1, wherein the at least one further air filter of the second filter stage is mounted in the air duct such that the at least one further air filter is completely present inside the air guiding interior part of the air duct, independently of the state of the filter and there is no bulge in the air duct for accommodating the at least one further air filter and/or there is no bypass for bypassing the at least one further air filter in the air duct.

16. (canceled)

17. At least one of an air handling unit, rooftop unit, fan coil, heating system, ventilating system and an air-conditioning system comprising an air treatment device further comprising an air duct configured to deliver air flow; a first filter stage comprising at least one air filter and arranged in the air duct for filtering the air flow; at least a second filter stage comprising at least one further air filter and arranged downstream of the first filter stage for filtering the air flow after having passed the first filter stage; an air quality sensor is present, which allows for measuring the air quality of the air flow in between the first and the second air filter stages; and wherein the second filter stage is controllable between at least two states with different filtration efficiencies, such that, with respect to a filtration efficiency in the first state, a filtration efficiency in the second state is lower the filtration efficiency in the second state is essentially zero.

18. The at least one of an air handling unit, rooftop unit, fan coil, heating system, ventilating system and an air-conditioning system according to claim 17, further comprising an inlet configured to supply fresh air from outside of a building, to an upstream side of the first filter stage of the air treatment device and an outlet configured to discharge air having passed the air treatment device into the building.

19. (canceled)

20. A method for filtering air comprising the steps of:
a) delivering an air flow with an air duct having a first filter stage being arranged in the duct and comprising at least one air filter, and a second filter stage downstream of the first air filter stage comprising at least one further air filter; whereby the second air filter stage is controllable between at least two states with different filtration efficiencies, whereby, with respect to a filtration efficiency in the first state, a filtration efficiency in the second state is lower;
b) filtering the air flow with the first filter stage;
c) measuring the air quality in the air flow between the two filter stages with an air quality sensor;
d) comparing the reading of the air quality sensor with a given threshold and:
if the reading of the air quality sensor is equal to or above a threshold, bringing the second filter stage in a state with a filtration efficiency closer to the first state, switching the second filter stage to the first state or keeping it in the first state; or
if the reading of the air quality sensor is below the threshold, bringing the second filter stage in a state with a filtration efficiency closer to the second state, switching the second filter stage to the second state or keeping it in the second state.

21. The method according to claim 20, whereby the air flow in the duct is generated with a fluid-flow machine, and an air velocity of the air flow in the duct is determined with an air velocity probe, wherein the fluid-flow machine is controlled such that the air velocity of the air flow in the duct is essentially constant independent of the state of the second filter stage.

22. The method according to claim 20 wherein, based on the reading of the air quality sensor, the control of the second filter stage is set and adjusted.

23. The method according to claim 20, wherein, based on the reading of the air quality sensor of abnormalities which are detected, at least one of a threshold for controlling the second filter stage is determined and a time to maintenance for one of the air filters is predicted.

24. The method according to claim 20, wherein the air flow delivered in step a) comprises outside air from outside of a building, wherein the outside air delivered in step a) at first is filtered with the first filter stage before entering the building.
